(19) United States
(12) Patent Application Publication
(10) Pub. No.: US 2025/0205433 A1
(43) Pub. Date: Jun. 26, 2025

(54) ADJUSTABLE DEPTH AUTOINJECTOR

(71) Applicant: AMGEN INC., Thousand Oaks, CA (US)

(72) Inventors: Madeline Ann Mannion, Beverly, MA (US); Richard Lee Hellstrom, JR., Paso Robles, CA (US); Peter Dan Kaare Soelberg, Gentofte (DK)

(21) Appl. No.: 18/843,897

(22) PCT Filed: Mar. 8, 2023
(86) PCT No.: PCT/US23/14768

§ 371 Date: Sep. 4, 2024

Related U.S. Application Data
(60) Provisional application No. 63/319,049, filed on Mar. 11, 2022.

(51) Int. Cl. A61M 5/32; A61M 5/46

(52) U.S. Cl. CPC A61M 5/3245; A61M 5/46; A61M 2005/3247

(57) ABSTRACT

A drug delivery device includes a housing having proximal and distal ends and a longitudinal axis extending therebetween, an injection assembly at least partially disposed within the housing at or near the proximal end thereof and including a needle or a cannula, a shield slidably coupled with the housing, a drive assembly at least partially disposed within the housing, and a depth adjuster operably coupled with the shield and/or the housing. The shield is positionable in an extended position in which at least a proximal end extends a distance beyond the proximal end of the housing and a retracted position. The drive assembly is operably coupled with the injection assembly and the shield and is engageable to deliver a medicament via the injection assembly. When coupled with the shield and/or the housing, the depth adjuster is adapted to prevent or resist the shield from being positionable in the retracted position.

### CROSS-REFERENCE TO RELATED APPLICATION

Priority is claimed to U.S. Provisional Patent Application No. 63/319,049, filed Mar. 11, 2022, the entire contents of which are hereby incorporated by reference herein.

### FIELD OF DISCLOSURE

The present disclosure generally relates to drug delivery devices, and, more particularly, to adjustable features for drug delivery devices.

### BACKGROUND

Drug delivery devices, such as injectors, are used to deliver liquid drugs to a patient. Upon activation, a drug delivery device will expel a drug stored within an internal reservoir such as, for example, a pre-filled syringe (“PFS”) through a needle, cannula, or other delivery member into the patient. Some drug delivery devices, such as pen-type autoinjectors or on-body injectors, may be positioned adjacent to a patient's skin to deliver a drug via an injection needle or some other means over a period of time. The drug delivery device may be positioned near the tissue of the patient's abdomen, thigh, arm, or some other portion of the patient's body.

When using such devices, the delivery member is inserted a desired depth to provide subcutaneous or intramuscular drug delivery. Because children and other patients continue to grow, it is the appropriate delivery depth for particular drugs may change. For example, children's skin is oftentimes more thin than adults, and as such, the subcutaneous depth is not as deep. Devices that accommodate children of various ages and sizes are typically not used due in part to high development costs and the variety of devices needed to address changing sizes of children as they grow. Further, adult-designated devices are oftentimes not used for pediatric patients due to their increased needle penetration depths, as such devices may potentially deliver the drug to undesirable depths. As such, children are typically administered drugs in pre-filled syringes as opposed to devices such as autoinjectors that may incorporate more patient-friendly features.

The present disclosure sets forth adjustable depth autoinjectors and corresponding approaches embodying advantageous alternatives to existing devices and approaches, and that may address one or more of the challenges or needs mentioned herein.

### SUMMARY

In accordance with a first aspect, a drug delivery device is provided that includes a housing having proximal and distal ends and a longitudinal axis extending therebetween, an injection assembly at least partially disposed within the housing at or near the proximal end thereof and including a needle or a cannula, a shield slidably coupled with the housing, a drive assembly at least partially disposed within the housing, and a depth adjuster operably coupled with the shield and/or the housing. The shield is positionable in an extended position in which at least a proximal end extends a distance beyond the proximal end of the housing and a retracted position. The drive assembly is operably coupled with the injection assembly and the shield and is engageable to deliver a medicament via the injection assembly. When coupled with the shield and/or the housing, the depth adjuster is adapted to prevent or resist the shield from being positionable in the retracted position.

In some examples, the depth adjuster includes a collar having an opening to receive a portion of the shield and a body. The body of the collar may engage a portion of the shield and a portion of the housing to prevent or resist relative movement between the shield and the housing. In some examples, the proximal end of the shield includes a ridge that engages a portion of the collar. Further, in some examples, the body may have a thickness between approximately 1 mm and approximately 5 mm. In some approaches, the depth adjuster is selectable between a plurality of depth adjusters having varying thicknesses.

In these and other examples, the depth adjuster includes a rotatable knob that selectively engages a portion of the shield. Further, the rotatable knob may be disposed on a threaded member of the housing. In some examples, the rotatable knob may include at least one linear step to adjust a point of engagement with the portion of the shield.

In accordance with a second aspect, a drug delivery device is provided that includes a housing having proximal and distal ends and a longitudinal axis extending therebetween, an injection assembly at least partially disposed within the housing at or near the proximal end thereof and including a needle or a cannula, and a drive assembly at least partially disposed within the housing and operably coupled with the injection assembly. The drive assembly is engageable to deliver a medicament via the injection assembly. The housing is adapted to slidably receive at least one of: 1) a first shield having a first configuration; or 2) a second shield having a second configuration that is different than the first configuration of the first shield. The first shield is adapted to cause the needle or cannula to extend a first length from a proximal end thereof, and the second shield is adapted to cause the needle or cannula to extend a second length from a proximal end thereof.

In accordance with a third aspect, a platform system for assembling a drug delivery device is provided that includes a housing having proximal and distal ends and a longitudinal axis extending therebetween, an injection assembly at least partially disposed within the housing at or near the proximal end thereof and including a needle or a cannula, and a drive assembly at least partially disposed within the housing and operably coupled with the injection assembly. The drive assembly is engageable to deliver a medicament via the injection assembly. The system further includes a group of selectable depth adjustment components adapted to couple with a portion of the drug delivery device to at least partially restrict movement of a portion of the injection assembly. The drug delivery device is assembled by using at least one desired characteristic of the drug delivery device to identify and select a first depth adjustment component from the group of selectable depth adjustment components and coupling the first depth adjustment component with at least one of the housing, the injection assembly, or the drive assembly.

### BRIEF DESCRIPTION OF THE DRAWINGS

The above needs are at least partially met through provision of the adjustable depth autoinjector described in the following detailed description, particularly when studied in conjunction with the drawings, wherein:

FIG. 1 illustrates a cross-sectional view an example drug delivery device in accordance with various embodiments;

FIG. 2 illustrates the example drug delivery device of FIG. 1 having an example depth adjuster coupled therewith in accordance with various embodiments;

FIG. 3 illustrates the example drug delivery device of FIG. 2 in accordance with various embodiments;

FIG. 4 illustrates the example drug delivery device of FIGS. 2 and 3 having a number of varying example depth adjusters coupled therewith in accordance with various embodiments;

FIG. 5 illustrates a front view of the example depth adjuster of FIGS. 2-4 in accordance with various embodiments;

FIG. 6 illustrates an example side view of the example depth adjuster of FIGS. 2-5 in accordance with various embodiments;

FIG. 7 illustrates a front view of an alternative example depth adjuster in accordance with various embodiments;

FIG. 8 illustrates an example side view of the alternative example depth adjuster of FIG. 7 in accordance with various embodiments;

FIG. 9 illustrates an example drug delivery device having an alternative depth adjuster in the form of a knob in accordance with various embodiments;

FIG. 10 illustrates a cross-sectional view of the example drug delivery device of FIG. 9 in accordance with various embodiments;

FIG. 11 illustrates a cross-sectional view of the example drug delivery device of FIG. 9 having an alternative depth adjuster in the form of a knob having a different configuration in accordance with various embodiments;

FIG. 12 illustrates an example drug delivery device having a first example depth adjustment mechanism in the form of a shield having a first characteristic in accordance with various embodiments;

FIG. 13 illustrates a cross-sectional view of the example drug delivery device of FIG. 12 in accordance with various embodiments;

FIG. 14 illustrates a cross-sectional view of an example drug delivery device having a second example depth adjustment mechanism in the form of a shield having a second characteristic in accordance with various embodiments;

FIG. 15 illustrates a cross-sectional view of an example drug delivery device having a third example depth adjustment mechanism in the form of a shield having a third characteristic in accordance with various embodiments;

FIG. 16 illustrates a cross-sectional view of an example drug delivery device having a fourth example depth adjustment mechanism in the form of a shield having a fourth characteristic in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, systems and approaches are provided that adjust an insertion depth of a drug delivery device such as, for example, an autoinjector. The systems and approaches described herein may provide for a range of insertion depth adjustability between approximately 1 mm and approximately 15 mm. Further, in some examples, such a system may be provided as a kit where a number of depth adjusting components may be provided that a user may quickly select between based on the desired drug administration profile (e.g., based on the patient's age, weight, body mass index, and/or other factors). Such a system advantageously may not require significant component redesign and/or a rearrangement of components, and as such, existing drug delivery devices may be easily and readily retrofitted as desired.

The drug delivery device delivers a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state. Various implementations and configurations of the drug delivery device are possible. For example, the present disclosure describes a drug delivery device in the form of a single-use, disposable injector. In other embodiments, the drug delivery device may be configured as multiple-use reusable injector. The drug delivery device may be operable for self-administration by a patient or for administration by a caregiver or a formally trained healthcare provider (e.g., a doctor or nurse). Further, the drug delivery device may take the form of an autoinjector or pen-type injector, and, as such, may be held in the hand of the user over the duration of drug delivery or dosing.

Turning to the Figures, and with particular reference to FIGS. 1-7, a drug delivery device 10 for delivering a drug, which may also be referred to herein as a medicament or drug product, is provided. The drug may be, but is not limited to, various biologicals such as peptides, peptibodies, or antibodies. The drug may be in a fluid or liquid form, although the disclosure is not limited to a particular state. In certain liquid formulations, the drug may have a viscosity between approximately (e.g., ±10%) 1-13 centipoise (cP), approximately (e.g., ±10%) 1-30 cP, approximately (e.g., ±10%) 1-60 cP, or other suitable viscosity profiles. Other examples are possible.

Various implementations and configurations of the drug delivery device 10 are possible. For example, the present disclosure describes a drug delivery device 10 is in the form of a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). Further, in the illustrated examples, the drug delivery device 10 takes the form of an autoinjector or pen-type injector, and, as such, may be held in the hand of the user over the duration of drug delivery or dosing.

The drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis “A” between a proximal end 12a and a distal end 12b. The drug delivery device 10 further includes an injection assembly 15 and a drive assembly 30. The injection assembly 15 and the drive assembly 30 may each be at least partially disposed within the housing 12. The injection assembly 15 includes a delivery member 16 in the form of a needle or a cannula. An opening 14 may be formed in the proximal end 12a to permit an insertion end 16a of the delivery member 16 to extend outside of (i.e., beyond the length of) the housing 12.

The injection assembly 15 may further include a drug storage container 23 which may be disposed within an interior space of the housing 12 and is configured to contain a drug 24. The drug storage container 23 may be pre-filled and shipped, e.g., by a manufacturer, to a location where the drug storage container 23 is combined with a remainder of the drug delivery device 10. The housing 12 may be pre-loaded with the drug storage container 23, e.g., by a manufacturer, or alternatively, loaded with the drug storage container 23 by a user prior to use of the drug delivery device 10. The drug storage container 23 may include a rigid wall defining an internal bore or reservoir. The wall may be made of glass or plastic. Proximal movement of a stopper or similar device may expel the drug 24 from the reservoir of the drug storage container 23 into the delivery member 16. The distal end of the drug storage container 23 may be open to allow a plunger to extend into the drug storage container 23 and push the stopper in the proximal direction. Upon activation of the drive assembly 30, the plunger may move in the proximal direction to drive the stopper in the proximal direction.

The delivery member 16 is connected or operable to be connected in fluid communication with the reservoir of the drug storage container 23. A proximal end of the delivery member 16 may define the insertion end 16a of the delivery member 16. The insertion end 16a may include a sharpened tip of other pointed geometry allowing the insertion end 16a to pierce the patient's skin and subcutaneous tissue during insertion of the delivery member 16. The delivery member 16 may be hollow and have an interior passageway. One or more openings may be formed in the insertion end 16a to allow drug to flow out of the delivery member 16 into the patient.

In the present embodiment, the drug storage container 23 is a pre-filled syringe and has a staked, hollow metal needle for the delivery member 16. Here, the needle is fixed relative to the wall of the drug storage container 23 and is in permanent fluid communication with the reservoir of the drug storage container 23. In other embodiments, the drug storage container 23 may be a needle-less cartridge, and, as such, initially may not be in fluid communication with the delivery member 16. In such embodiments, the drug storage container 23 may move toward a distal end of the delivery member 16, or vice versa, during operation of the drug delivery device 10 such that the distal end of the delivery member 16 penetrates through a septum covering an opening in the drug storage container 23 thereby establishing fluid communication with the reservoir of the drug storage container 23.

The drug storage container 23 may be fixed relative to the housing 12 such that the drug storage container 23 does not move relative to the housing 12 once installed therein. As such, the insertion end 16a of the delivery member 16 may extend permanently through the opening 14 in the housing 12 in the pre-delivery, delivery, and post-delivery states. In alternative embodiments, the drug storage container 23 may be moveably coupled to the housing 12 such that the drug storage container 23 is able to move relative to the housing 12 during operation of the drug delivery device 10. In certain such alternative embodiments, the insertion end 16a of the delivery member 16 may be retracted within the opening 14 in the housing 12 in the pre-delivery state. Subsequently, during operation of the injection device 10, the insertion end 16a of the delivery member 16 may be deployed through the opening 14 in the housing 12 for insertion into the patient. This motion may, in some embodiments, be the result of the drug storage container 23 having been driven in the proximal direction relative to the housing 12.

As previously noted, the drug delivery device 10 may further include the drive assembly 30 disposed partially or entirely within the housing 12. Generally, the drive assembly 30 may be configured to store energy and, upon or in response to activation of the drive assembly 30 by the user, release or output that energy to drive the injection assembly 15 (i.e., the delivery member 16, the drug storage container 23, etc.) to expel the drug 24 from the drug storage container 23 through the delivery member 16 into the patient. In the present example, the drive assembly 30 is configured to store mechanical potential energy; however, alternative embodiments of the drive assembly 30 may be configured differently, with, for example, the drive assembly 30 storing electrical or chemical potential energy. Upon activation of the drive assembly 30, the drive assembly 30 may convert the potential energy into kinetic energy for moving the plunger and other components.

In some examples, the drive assembly 30 may include a biasing member such as, for example, a rotational biasing member and additional components used to engage the biasing member to release its mechanical potential energy. In some implementations, the rotational biasing member may be a torsion spring (e.g., a spiral torsion, a helical torsion spring, etc.) which is initially retained in an energized state. In the energized state, the rotational biasing member may be twisted or wound and retained in that twisted or wound configuration by such additional components. When released, the rotational biasing member will try to return to its natural length or shape, and as a result, exert a biasing force causing the components to rotate, which in turn may convert the rotational motion into linear motion for driving the plunger in the proximal direction. Alternative embodiments may utilize an energy source different from a rotational biasing member. Certain alternative embodiments may utilize, for example, a linear biasing member (e.g., a helical compression spring, a helical extension spring, etc.) which, when released, outputs a force in the direction of travel of the plunger. In addition to or as an alternative to a biasing member, other embodiments may include any one or combination of: an electromechanical arrangement including an electric motor and/or solenoid and a drive train or transmission coupled to the plunger; or an arrangement that generates or releases a pressurized gas or fluid to propel the plunger or which acts directly on the stopper to move stopper through the drug storage container 23 to expel the drug 24 from therein. In embodiments where the drug storage container 23 and/or the delivery member 16 is moveable relative to the housing 12, the drive assembly 30 may, upon activation, drive the drug storage container 23 and/or the delivery member 16 in the proximal direction so as to cause the insertion end 16a of the delivery member 16 to be inserted into the patient. Thus, in certain embodiments, the drive assembly 30 may provide the motive force needed for both inserting the delivery member 16 into the patient and expelling the drug 24 from the drug storage container 23.

The drug delivery device 10 may further include a guard mechanism for preventing contact with the insertion end 16a of the delivery member 16 when the drug delivery device 10 is not being used to administer an injection. The guard mechanism may include a shield 60 moveably disposed at the proximal end 12a of the housing 12 adjacent to the opening 14. The shield 60 may have a hollow and generally cylindrical or tubular shape. The shield 60 may have a distal end received within the housing 12, and may be configured to move relative to the housing 12 between an extended position wherein a proximal end 60a of the shield 60 extends through the opening 14 in the housing 12 and a retracted position wherein the proximal end of the shield 60 is retracted, fully or partially, into the opening 14 in the housing 12. In at least the extended position, the shield 60 may extend beyond and surround the insertion end 16a of the delivery member 16. In some embodiments, moving the shield 60 toward the retracted position may expose the insertion end 16a of the delivery member 16. Further, in some embodiments, the shield 60 may be coupled to the housing 12 and/or the drive mechanism 30 such that the shield 60 is able to translate in a linear direction relative to the housing 12.

The proximal end of the shield 60 may include a skin contacting portion 62. The distal end of the shield 60 may include an activator or engagement portion 64. In some examples, the activator portion 64 and the skin-contacting portion 62 may be integrally formed to define a single, monolithic structure. At least the skin-contacting portion 62 of shield 60 may have a hollow and generally cylindrical or tubular shape and, in some embodiments, may be centered about the longitudinal axis A of the drug delivery device 10. The activator portion 64 of the shield 60 may be a cutout or recessed region.

Moving the shield 60 from the extended position to the retracted position may be accomplished by pressing the skin-contacting portion 62 against the patient's skin at the injection site. In examples where the delivery member 16 protrudes from the opening 14 in the housing 12 in the pre-delivery or storage state, this motion may result in the insertion end 16a of the delivery member 16 being inserted into the patient's skin.

It will be appreciated that the device 10 may include any number of additional components such as, for example, a guard biasing member (not illustrated) which may bias or urge the shield 60 towards the extended position by exerting a biasing force in the proximal direction thereon. In some examples, the guard biasing member is in the form of a compression spring. In other examples, the guard biasing member may be in the form of a torsion or other form of spring. In any event, a user may overcome this biasing force by pressing the shield 60 against the injection site with sufficient force. When the injection is complete and the drug delivery device 10 is lifted off of the injection site, the guard biasing member may return the shield 60 to the extended position, thereby covering the insertion end 16a of the deliver member 16.

The shield 60 may be configured to interact with the drive assembly 30 when the shield 60 moves from the extended position to the retracted position. This interaction may activate the drive assembly 30 to output the energy needed for driving the plunger to expel the drug 24 from the drug storage container 23 and/or insert the insertion end 16a of the delivery member 16 into the patient's skin. In some embodiments, movement of the shield 60 from the extended position to the retracted position releases the rotational biasing member from the energized state, thereby allowing the rotational biasing member to de-energize and drive the plunger to expel the drug 24 from the drug storage container 23. When the shield 60 moves from the extended position to the retracted position as a result of being pressed against the patient's skin, the activator portion 64 of the shield 60 engages the drive assembly 30 to release the rotational biasing member and drive the plunger in the proximal direction.

Notably, when the shield 60 is fully moved to the retracted position, the delivery member 16 extends and/or protrudes a maximum distance from the skin contacting portion 62. In some examples, this distance may be between approximately 10 mm and 15 mm, though other examples are possible. However, the desired delivery site for some patients (e.g., children) may be a lesser depth than this maximum value (e.g., between approximately 3 mm and 8 mm).

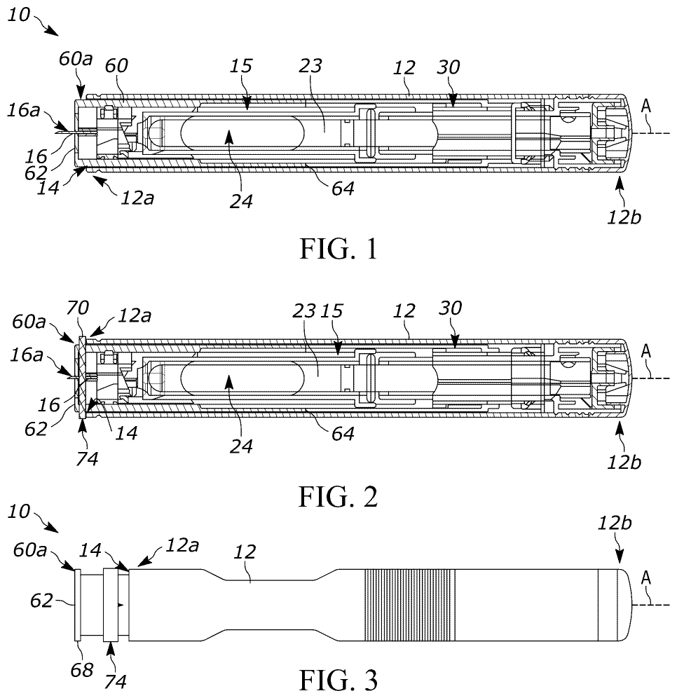

To achieve drug delivery at relatively lesser depths, the drug delivery device 10 further includes a depth adjuster 70. In the illustrated examples of FIGS. 2-8, the depth adjuster 70 is in the form of a collar having an opening 72 and a body 74. The body 74 of the depth adjuster 70 may have any number of desired thicknesses such as, for example, between approximately 1 mm and 15 mm. The opening 72 of the collar 70 may operably engage and/or receive a portion of the shield 60, and as such, may be movable with the shield 60 relative to the housing 12. In other examples (not illustrated, the depth adjuster 70 may be operably coupled with the housing 12. As illustrated in FIGS. 5-8, the depth adjuster 70 may have any number of cross-sectional shapes (e.g., a generally circular member, a C-shaped member, etc.).

The shield 60 may further define an abutment surface or ridge 68 which may engage and/or retain the body 74 of the collar 70. Further, an opposing end of the body 74 may engage the proximal end 12a of the housing 12. So arranged, and as illustrated in FIG. 2, the collar 70 acts as a wedge to prevent or resist the shield 60 from fully moving to the retracted position. By preventing or resisting the shield 60 from fully moving to the retracted position, less than the entire length of the delivery member 16 will be exposed and able to be inserted into the patient, and as such, the delivery member 16 will extend a lesser depth into the patient, which may be desirable for certain patients such as children. In these and other implementations, the thickness of the body 74 of the collar 70 is directly correlated to the reduction in depth the delivery member 16 may reach. For example, if the body 74 of the collar 70 is approximately 5 mm thick, the resulting insertable length of the delivery member 16 is reduced by approximately 5 mm.

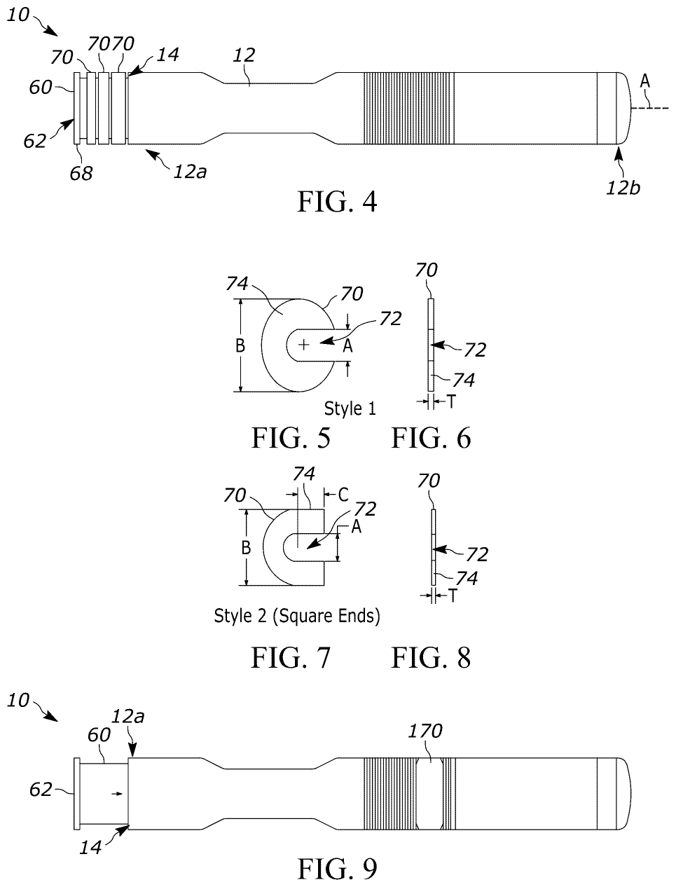

As previously noted, the depth adjuster 70 may have any number of arrangements and/or thicknesses. In some examples, a kit or system may be provided with the device 10 that includes multiple depth adjusters 70 having varying thicknesses. For example, the kit may include a first depth adjuster 70 having a thickness of approximately 1 mm, a second depth adjuster 70 having a thickness of approximately 2 mm, a third depth adjuster 70 having a thickness of approximately 3 mm, and so on. In some examples, each different depth adjuster 70 may have a different visual characteristic such as color, texture, text, etc. to allow a user to quickly identify the thickness of the depth adjuster 70. So configured, a user (e.g., a primary care physician or other person responsible with administering the device 10) may select a desired depth adjuster or adjusters 70 from the kit that are suitable for properly administering the device 10 to the patient at the desired delivery depth. As illustrated in FIG. 4, in some examples, multiple depth adjusters 70 may be coupled with the shield 60 as desired to reduce the overall distance the delivery member 16 may extend.

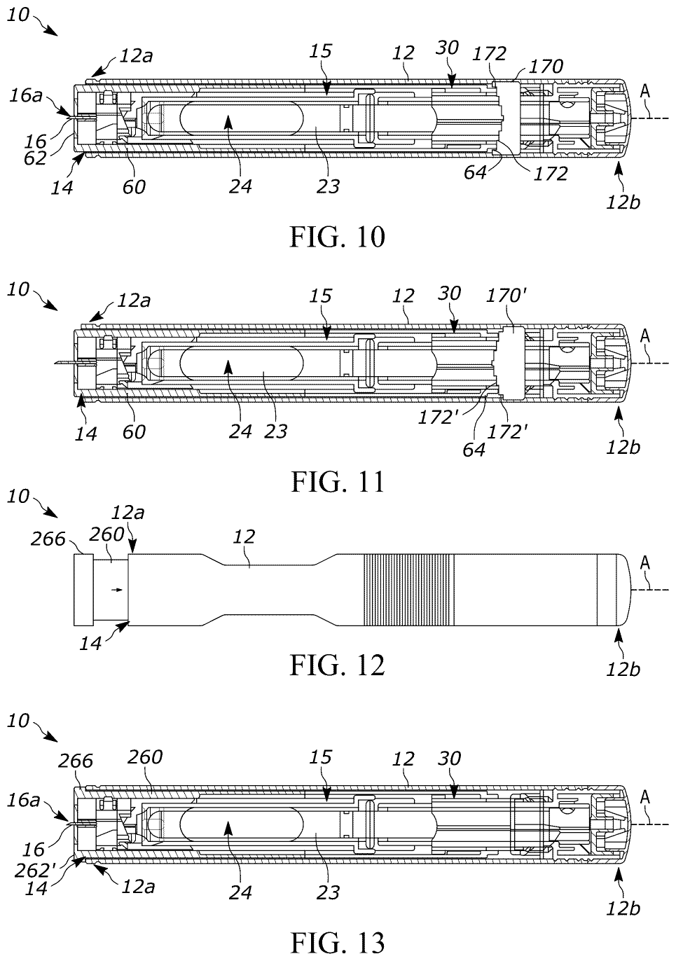

With reference to FIGS. 9-11, an alternative depth adjuster 170 may be provided for use with the drug delivery device 10. In these examples, the depth adjuster 170 is in the form of a rotatable knob having a number of steps 172. The rotatable knob 170 may be coupled with or otherwise positioned on the housing 12. For example, the housing 12 may include a cutout portion through which a portion of the rotatable knob 170 may extend. In some examples, the housing 12 may include a threaded region that engages a corresponding threaded portion of the rotatable knob 170. As illustrated in FIGS. 10 and 11, a portion of the shield 60 (e.g., the engagement portion 64) may be positioned a distance from the rotatable knob 170 while the shield 60 is in the extended position. However, upon pressing the skin contacting portion 62 of the shield 60 against a users' skin to begin the drug administration process, the engagement portion 64 of the shield 60 may contact the rotatable knob ad a specified step 172, thus limiting axial movement of the shield 60. As a result, a reduced length of the delivery member 16 will be exposed and accordingly will extend a lesser depth into the patient when delivering the drug.

The user may selectively rotate the rotatable knob 170 to cause a different step 172 to engage the shield during drug administration. As illustrated in FIG. 10, the steps 172 are arranged in a generally concave arrangement, and in FIG. 11, which depicts an alternative rotatable knob 170′, the steps 172′ are arranged in a generally convex arrangement. In some examples (not illustrated), the rotatable knob 170 may include a number of different visual indicators to assist with identifying the depth at which the delivery member 16 will be inserted.

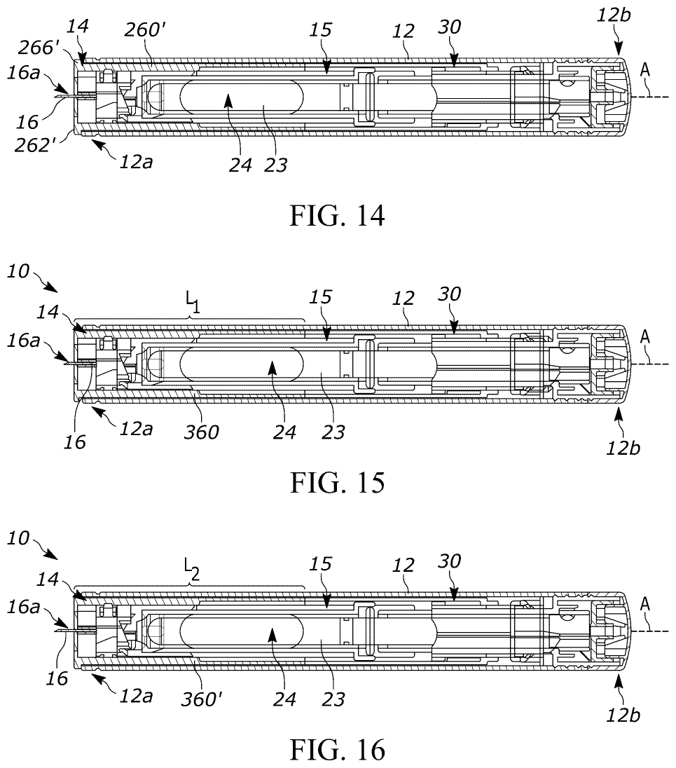

With reference to FIGS. 12-14, the drug delivery device 10 is provided with yet another alternative mechanism to adjust the insertion depth of the delivery member 16. In these examples, alternative shields 260, 260′ are provided. These shields 260, 260′ may have similar features as the shield 60, and as such, will not be described in substantial detail. Each of the shields 260, 260′ may have different configurations that cause the delivery member 16 to be inserted at different depths. More specifically, the shield 260 illustrated in FIG. 13 includes a lip 266 having a first thickness, and the shield 260′ illustrated in FIG. 14 includes a lip 266′ having a second thickness. These lips 266, 266′ cause the delivery member 16 to extend from the respective shields 260, 260′ at varying lengths. Notably, the lip 266 illustrated in FIG. 13 is relatively wider, and as such, reduces the insertion depth of the delivery member a greater amount from the maximum value as compared with the relatively narrow lip 266′ illustrated in FIG. 14, which reduces the insertion depth of the delivery member 16 a lesser amount from the maximum value. It will be appreciated that like the depth adjusters 70, a kit or system may be provided having any number of shields having lips of varying thicknesses.

With reference to FIGS. 15 and 16, the drug delivery device 10 is provided with yet another alternative mechanism to adjust the insertion depth of the delivery member 16. In these examples, alternative shields 360, 360′ are provided. These shields 360, 360′ may have similar features as the shields 60, 260, 260′, and as such, will not be described in substantial detail. Each of the shields 360, 360′ may have different configurations that cause the delivery member 16 to be inserted at different depths. More specifically, the shield 360 illustrated in FIG. 15 has a first overall length, and the shield 360′ illustrated in FIG. 16 has a second overall length. These different lengths cause the delivery member 16 to extend from the respective shields 360, 360′ at varying lengths. Notably, the length of the shield 360 illustrated in FIG. 15 is relatively longer, and as such, the shield 360 reduces the insertion depth of the delivery member a greater amount from the maximum value as compared with the relatively shorter shield 360′ illustrated in FIG. 16, which reduces the insertion depth of the delivery member 16 a lesser amount from the maximum value. It will be appreciated that like the depth adjusters 70 and shields 260, 260′, a kit or system may be provided having any number of shields having varying lengths.

In some examples, a platform system or kit may be provided for assembling the drug delivery device 10. The platform system includes a group of selectable depth adjustment components that couple with the device 10 to restrict relative movement of a portion of the injection assembly. The device 10 is assembled by using any number of desired characteristics (such as, for example, the desired injection depth) of the drug delivery device to identify and select a first, desired depth adjustment component from the group of selectable depth adjustment components and coupling the first depth adjustment component with at least one of the housing, the injection assembly, or the drive assembly. For example, the group of selectable depth adjustment components may include the varying depth adjusters 70, the varying rotatable knobs 170, and/or the varying shields 260, 260′, 360, 360′. It will be appreciated that in examples where the desired depth adjustment component is one of the depth adjusters 70 and/or rotatable knobs 170, the device 10 will additionally include the shield 60, but in examples where the desired depth adjustment component is one of the shields 260, 260′, 360, 360′, these shields will replace the shield 60 in the device. It is appreciated that any combination of the depth adjustment components may be used together as desired. So configured, a user (e.g., a primary care physician or other person responsible with administering the device 10) may select a desired depth adjuster 70 suitable for properly administering the device 10 to the patient.

In some examples, the depth adjustment components described herein may also be coupled with components that limit movement of the storage unit, and may be positioned at different locations within the device 12. For example, the depth adjustment component may also limit the plunger depth. In such examples, varying plunger rods having different lengths may be provided as needed.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNa mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing having a proximal end, a distal end, and a longitudinal axis extending between the proximal end and the distal end thereof;
an injection assembly at least partially disposed within the housing at or near the proximal end thereof, the injection assembly including a needle or a cannula;
a shield slidably coupled with the housing, the shield being positionable in an extended position in which at least a proximal end of the shield extends a distance beyond the proximal end of the housing and a retracted position;
a drive assembly at least partially disposed within the housing and operably coupled with the injection assembly and the shield, the drive assembly engageable to deliver a medicament via the injection assembly; and
a depth adjuster operably coupled with at least one of the shield or the housing;
wherein when the depth adjuster is coupled with at least one of the shield or the housing, the depth adjuster is adapted to prevent or resist the shield from being positionable in the retracted position.

2. The drug delivery device of claim 1, wherein the depth adjuster comprises a collar having an opening to receive a portion of the shield and a body, the body of the collar adapted to engage a portion of the shield and a portion of the housing to prevent or resist relative movement between the shield and the housing.

3. The drug delivery device of claim 2, wherein the proximal end of the shield includes a ridge that engages a portion of the collar.

4. The drug delivery device of claim 2, wherein the body has a thickness between approximately 1 mm and approximately 5 mm.

5. The drug delivery device of claim 1, wherein the depth adjuster is selectable between a plurality of depth adjusters having varying thicknesses.

6. The drug delivery device of claim 1, wherein the depth adjuster comprises a rotatable knob that selectively engages a portion of the shield.

7. The drug delivery device of claim 6, wherein the rotatable knob is disposed on a threaded member of the housing.

8. The drug delivery device of claim 6, wherein the rotatable knob includes at least one linear step to adjust a point of engagement with the portion of the shield.

9. A drug delivery device comprising:
a housing having a proximal end, a distal end, and a longitudinal axis extending between the proximal end and the distal end thereof;
an injection assembly at least partially disposed within the housing at or near the proximal end thereof, the injection assembly including a needle or a cannula;
a drive assembly at least partially disposed within the housing and operably coupled with the injection assembly, the drive assembly engageable to deliver a medicament via the injection assembly;
the housing adapted to slidably receive at least one of: 1) a first shield having a first configuration; or 2) a second shield having a second configuration that is different than the first configuration of the first shield;
wherein the first shield is adapted to cause the needle or cannula to extend a first length from a proximal end thereof and the second shield is adapted to cause the needle or cannula to extend a second length from a proximal end thereof.

10. The drug delivery device of claim 9, wherein the first shield has a first overall length, and the second shield has second overall length that is different than the first overall length.

11. The drug delivery device of claim 9, wherein the first shield includes a proximal end having a lip having a first thickness, and the second shield includes a proximal end having a lip having a second thickness that is different than the first thickness.

12. A platform system for assembling a drug delivery device, the system comprising:
a housing having a proximal end, a distal end, and a longitudinal axis extending between the proximal end and the distal end thereof;
an injection assembly at least partially disposed within the housing at or near the proximal end thereof, the injection assembly including a needle or a cannula;
a drive assembly at least partially disposed within the housing and operably coupled with the injection assembly, the drive assembly engageable to deliver a medicament via the injection assembly; and
a group of selectable depth adjustment components adapted to couple with a portion of the drug delivery device to at least partially restrict relative movement of a portion of the injection assembly;
wherein the drug delivery device is assembled by using at least one desired characteristic of the drug delivery device to identify and select a first depth adjustment component from the group of selectable depth adjustment components and coupling the first depth adjustment component with at least one of the housing, the injection assembly, or the drive assembly.

13. The platform system of claim 12, wherein the at least one desired characteristic comprises a desired injection depth.

14. The platform system of claim 12, wherein the group of selectable depth adjustment components comprises a plurality of shields, each of the plurality of shields having a different configuration.

15. The platform system of claim 14, where each of the plurality of shields has a different overall length.

16. The platform system of claim 14, wherein each of the plurality of shields includes a lip adapted to engage a portion of the housing, wherein each lip of the plurality of shields has a different thickness.

17. The platform system of claim 12, wherein the group of selectable depth adjustment components includes a plurality of depth adjusters operably coupled with a portion of the device.

18. The platform system of claim 17, wherein each of the plurality of depth adjusters comprises a collar having a body having a different thickness.
